(19) United States
(12) Patent Application Publication
(10) Pub. No.: US 2026/0363628 A1
(43) Pub. Date: Nov. 19, 2026

(54) GLUCOSE MANAGEMENT INDICATOR AS A PREDICTOR OF DIABETIC COMPLICATIONS

(71) Applicant: Abbott Diabetes Care Inc., Alameda, CA (US)

(72) Inventors: Yongjin XU, San Ramon, CA (US); Timothy Christian DUNN, San Francisco, CA (US)

(21) Appl. No.: 19/632,949

(22) Filed: Mar. 30, 2026

Related U.S. Application Data
(60) Provisional application No. 63/808,308, filed on May 19, 2025.

(51) Int. Cl. A61B 5/00; A61B 5/145; G16H 10/60; G16H 40/67

(52) U.S. Cl. CPC A61B 5/7275; A61B 5/0022; A61B 5/14532; A61B 5/6801; A61B 5/746; G16H 10/60; G16H 40/67

(57) ABSTRACT

Various embodiments relate to a computer-implemented method for managing diabetes, involving obtaining continuous glucose monitoring (CGM) data from an analyte sensor worn by an individual. The method includes calculating a glucose management indicator (GMI) derived from the average sensor glucose, a glycation ratio, and the glucose affinity of glucose transporter type-1 on red blood cells. A prediction for developing diabetic complications is generated based on the GMI. Actions initiated based on the prediction include modifying the individual's glycemic treatment plan, initiating alerts to personal or healthcare provider devices, and integrating the prediction into the individual's electronic health record.

### CROSS-REFERENCE TO RELATED APPLICATIONS

This application claims priority to, and the benefit of, U.S. Provisional Application No. 63/808,308, filed May 19, 2025, and entitled “GLUCOSE MANAGEMENT INDICATOR AS A PREDICTOR OF DIABETIC COMPLICATIONS,” the entire contents of which are incorporated by reference herein in their entirety.

### TECHNICAL FIELD

The subject matter described herein relates generally to improved analyte monitoring systems, as well as methods and devices relating thereto.

### BACKGROUND

The detection and/or monitoring of analyte levels, such as glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc., or the like, can be important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

During normal circulation of red blood cells in a mammal such as a human body, glucose molecules attach to hemoglobin, which is referred to as glycosylated hemoglobin (also referred to as glycated hemoglobin). The higher the amount of glucose in the blood, the higher the percentage of circulating hemoglobin molecules with glucose molecules attached. The level of glycosylated hemoglobin is increased in the red blood cells of subjects with poorly controlled diabetes mellitus. Since glucose molecules stay attached to hemoglobin for the life of the red blood cells (normally no more than about 120 days), the level of glycosylated hemoglobin reflects an average blood glucose level over that period.

Most of hemoglobin is a type called HbA. When glucose molecules attach to HbA molecules, glycosylated HbA is formed, which is referred to as HbA1. HbA1 has three components: HbA1a, HbA1b, and HbA1c. Because a glucose molecule binds more strongly and to a higher degree to HbA1c than HbA1a and HbA1b, a measure of HbA1c in blood (HbA1c test) is often used as an indication of a subject's average blood glucose level over a 120-day period (the average lifetime of a red blood cell), weighted more toward recent glucose levels. The HbA1c test is performed by drawing a blood sample from a subject at a medical professional's office, which is then analyzed in a laboratory. The HbA1c test may be used as a screening and diagnostic test for pre-diabetes and diabetes. The HbA1c test may be conducted multiple times over a time period to monitor the health of a subject for diagnosis and/or therapy decisions.

The subject matter claimed herein is not limited to embodiments that solve any disadvantages or that operate only in environments such as those described supra. Instead, this background is only provided to illustrate one example technology area where some embodiments described herein may be practiced.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to systems monitoring glucose, and in particular to predicting a diabetic complication based on a glucose management indicator (GMI) derived from continuous glucose monitoring (CGM) data.

The technical problem addressed by the disclosed subject matter is the limited predictive accuracy and clinical utility of conventional GMI calculations, which often deviate from actual HbA1c values. This limitation reduces the effectiveness of CGM data in predicting diabetic complications such as diabetic retinopathy. The disclosed systems and methods solve this problem by introducing a new GMI model that incorporates physiological parameters, such as a reference personal glycation ratio and glucose transporter affinity, to improve prediction accuracy and facilitate timely, data-driven interventions.

In some aspects, the techniques described herein relate to a method, implemented in a computer system, including: obtaining CGM data from an analyte sensor device worn by an individual; calculating a GMI based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M}); generating a prediction for developing a diabetic complication based on the GMI; and initiating an action based on the prediction, wherein the action is selected from a group consisting of: modifying a glycemic treatment plan for the individual, initiating a first alert at a personal device associated with the individual, initiating a second alert at a healthcare provider device, and integrating the prediction into an electronic health record (EHR) associated with the individual. For example, a PGR_{ref} can be 65.1 ml/g, and K_{M} can take an experimentally measured value of 473 mg/dl.

In some aspects, the techniques described herein relate to a computer system, including: a processor; and a computer-readable storage medium that stores computer-executable instructions that are executable by the processor to at least: obtain CGM data from an analyte sensor device worn by an individual; calculate a GMI based on the CGM data, wherein the GMI is derived from an AG obtained from the CGM data, a PGR_{ref}, and a K_{M}; generate a prediction for developing a diabetic complication based on the GMI; and initiate an action based on the prediction, wherein the action is selected from a group consisting of: modifying a glycemic treatment plan for the individual, initiating a first alert at a personal device associated with the individual, initiating a second alert at a healthcare provider device, and integrating the prediction into an EHR associated with the individual.

In some aspects, the techniques described herein relate to a computer-readable storage medium that stores computer-executable instructions that are executable by a processor to at least: obtain CGM data from an analyte sensor device worn by an individual; calculate a GMI based on the CGM data, wherein the GMI is derived from an AG obtained from the CGM data, a PGR_{ref}, and a K_{M}; generate a prediction for developing a diabetic complication based on the GMI; and initiate an action based on the prediction, wherein the action is selected from a group consisting of: modifying a glycemic treatment plan for the individual, initiating a first alert at a personal device associated with the individual, initiating a second alert at a healthcare provider device, and integrating the prediction into an EHR associated with the individual.

This Summary introduces a selection of concepts in a simplified form that are further described below in the Detailed Description. This Summary is not intended to identify key features or essential features of the claimed subject matter, nor is it intended to be used to determine the scope of the claimed subject matter.

### BRIEF DESCRIPTION OF THE DRAWINGS

To describe how the advantages of the systems and methods described herein can be obtained, a more particular description of the embodiments briefly described supra is rendered by reference to specific embodiments thereof, which are illustrated in the appended drawings. These drawings depict only typical embodiments of the systems and methods described herein and are not, therefore, to be considered to be limiting in their scope. Systems and methods are described and explained with additional specificity and detail through the use of the accompanying drawings, in which:

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIG. 3 illustrates a summary comparison of the operating characteristic area under the curve values and mutual information scores for various metrics as they relate to quantifying the likelihood of incident diabetic retinopathy (DR).

FIGS. 4A and 4B illustrate example decision tree models and performance metrics for predicting incident DR.

FIG. 5 illustrates a flow chart of an example of a method for using a continuous glucose monitoring data-derived glucose management indicator to improve glycemic treatment outcomes.

### DETAILED DESCRIPTION

Growing clinical data demonstrate a strong correlation between the frequency of glucose monitoring and glycemic control. Despite such correlation, however, many individuals diagnosed with a diabetic condition do not monitor their glucose levels as frequently as they should due to a combination of factors, including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

There are various types of in vivo analyte monitoring systems. For example, “Continuous Analyte Monitoring” systems, such as “Continuous Glucose Monitoring” systems (CGMs), can transmit data from a sensor control device to a reader device (e.g., a dedicated device or a general-purpose device running a reader application) continuously or at a regular interval without prompting, e.g., automatically according to a schedule. As another example, “Flash Analyte Monitoring” systems, such as “Flash Glucose Monitoring” systems, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily interstitial fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices, such as personal computers, have also been utilized with or incorporated into in vivo and in vitro monitoring systems.

### Example Embodiment of In Vivo Analyte Monitoring System

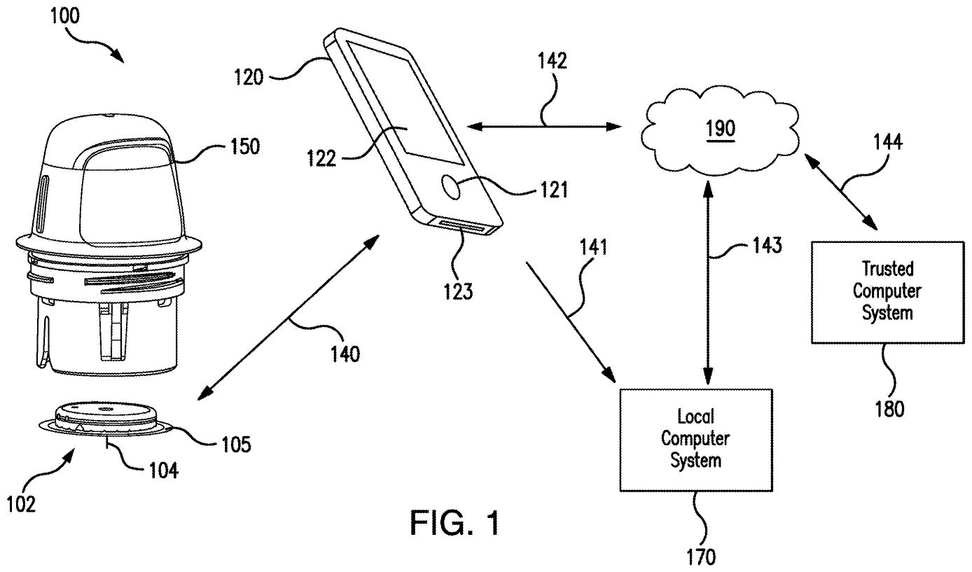

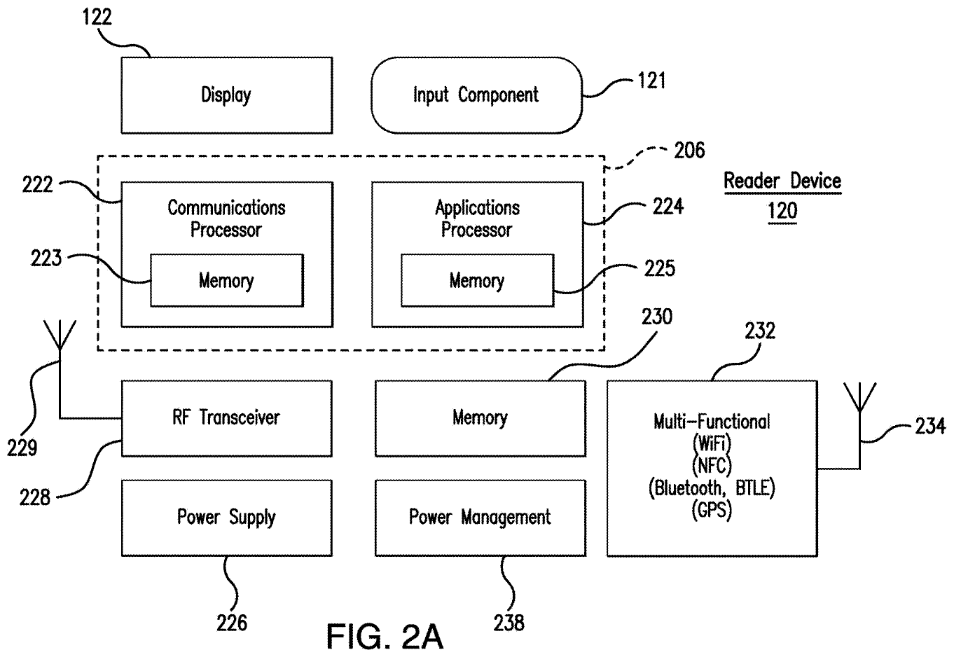

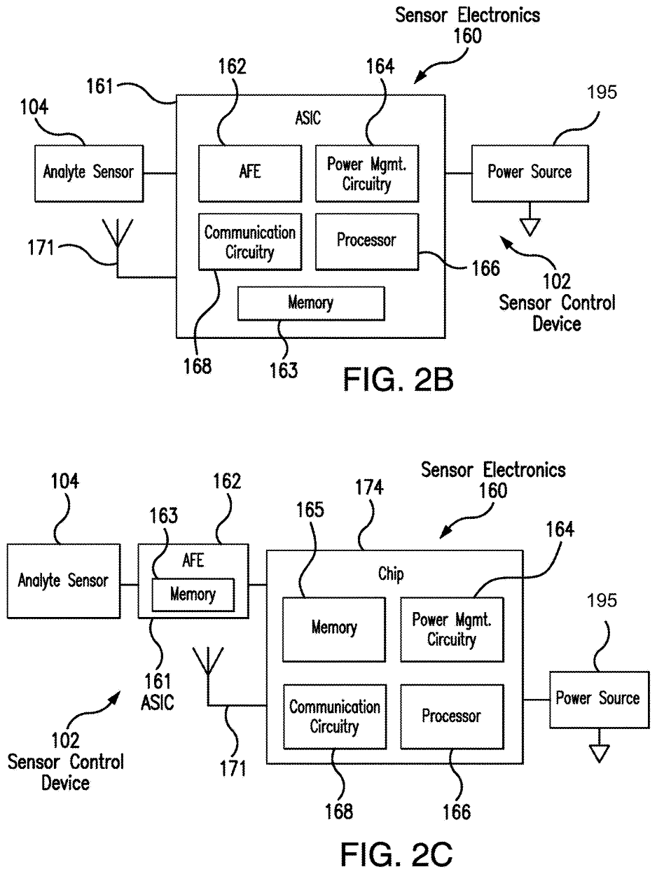

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where an analyte sensor 104 (e.g., an in vivo analyte sensor) is maintained in position, e.g., by an adhesive patch 105. Sensor control device 102 is further described in FIGS. 2B and 2C. Sensor control device 102 can communicate with the reader device 120 via a communication path 140 using a wired or wireless technique. Example wireless protocols include BLUETOOTH, BLUETOOTH LOW ENERGY (BLE, BTLE, BLUETOOTH SMART, etc.), Near Field Communication (NFC), and others. Users can view and use applications installed in memory on the reader device 120 using display 122 (which, in many embodiments, can comprise a touchscreen) and input component 121. A device battery of the reader device 120 can be recharged using a power port 123.

While FIG. 1 depicts only one reader device, in some embodiments, the sensor control device 102 can communicate with multiple reader devices. Each reader device 120 can communicate and share data with one another. In one example, reader devices include a smartphone and a smartwatch. In this example, the sensor control device 102 may communicate sensor data to the smartwatch, which then forwards it to the smartphone. Additionally, or alternatively, the sensor control device 102 may communicate sensor data to the smartphone, which then forwards it to the smartwatch. More details about reader device 120 are set forth with respect to FIG. 2A.

Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including BLUETOOTH, BLUETOOTH LOW ENERGY (BTLE), WIRELESS FIDELITY (Wi-Fi) or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth.

A trusted computer system 180 can include a cloud-based platform or server, and can provide for authentication services, secured data storage (e.g., storage of analyte measurement data received from one or more reader devices), report generation, and can communicate via communications path 144 with network 190 by wired or wireless technique. In addition, although FIG. 1 depicts trusted computer system 180 and local computer system 170 communicating with a single sensor control device (sensor control device 102) and a single reader device (reader device 120), it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in embodiments, can comprise a dedicated reader device, a smartphone, or a smart watch. Here, reader device 120 can include a display 122, an input component 121, and a processing core 206 including a communications processor 222 coupled with memory 223 and an applications processor 224 coupled with memory 225. Also included can be separate memory 230, radio frequency (RF) transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, BLUETOOTH, BTLE, and GPS with one or more antennas 234. As understood by one of skill in the art, these components are electrically and communicatively coupled in a manner to make a functional device.

### Example Embodiments of Sensor Control Devices

FIGS. 2B and 2C are block diagrams depicting example embodiments of a sensor control device 102 having an analyte sensor 104 and sensor electronics 160 (including analyte monitoring circuitry) that can have the majority of the processing capability for rendering end-result data suitable for display to the user. In FIG. 2B, a semiconductor chip 161 is depicted that, in some examples, can be a custom application-specific integrated circuit (ASIC). Shown within semiconductor chip 161 are certain high-level functional units, including an analog front end (AFE), AFE 162, power management circuitry 164 (or control circuitry), processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments, either circuit can perform the analyte monitoring function. Processor 166 can include one or more processors, microprocessors, controllers, and/or microcontrollers, each of which can be a discrete chip or distributed amongst (and a portion of) a number of different chips.

Semiconductor chip 161 also includes a memory 163 that can be shared by the various functional units present within semiconductor chip 161 or distributed amongst two or more of them. Memory 163 can also be a separate chip. Memory 163 can be volatile and/or non-volatile memory. In this embodiment, semiconductor chip 161 is coupled with power source 195, which can be a coin cell battery or the like. AFE 162 interfaces with analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed, in whole or in part, by processing circuitry on sensor control device 102, reader device 120, local computer system 170, or trusted computer system 180.

FIG. 2C is similar to FIG. 2B but instead includes two discrete semiconductor chips, AFE 162 and chip 174, which can be packaged together or separately. Here, AFE 162 is resident in semiconductor chip 161. Processor 166 is integrated with power management circuitry 164 and communication circuitry 168 on chip 174. AFE 162 may include memory 163, and chip 174 may include memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Using CGM Data to Estimate the Likelihood of Incident Diabetic Retinopathy (DR)

While CGMs have proven to be an indispensable tool for monitoring and managing an individual's blood glucose levels throughout each day, HbA1c still remains an important indicator of the individual's overall glucose levels over time, and thus for driving robust glycemic management plans for individuals with diabetes. In particular, a diabetic individual's HbA1c levels are recognized as being predictive of that individual's likelihood of developing incident complications of diabetes mellitus, such as DR, nephropathy, cardiovascular disease, and neuropathy. However, due to the cost and inconvenience of obtaining HbA1c tests, which generally involves a blood draw and laboratory analysis, many diabetic individuals do not obtain HbA1c tests at a frequency that adequately informs a robust glycemic management plan. In many cases, this leads to reduced glycemic control over time and increased instances of those individuals developing incident complications such as DR, nephropathy, cardiovascular disease, and neuropathy.

The glucose management indicator (GMI) is a glycemic metric, derived from CGM-derived measurements of average sensor glucose (AG), that aims to reflect a patient's actual HbA1c levels. A conventional GMI calculation was derived from linear regression analysis of AG and HbA1c data, and is shown in Equation 1:

GMI(NGSP %)=0.02392*AG (mg/dl)+3.31 Equation 1

where AG is measured in mg/dl (milligrams per deciliter). While proving useful for overall estimations of a patient's actual HbA1c levels, significant deviations in the agreement of GMI with measured HbA1c have been observed, particularly in the low (e.g., less than about 6.5%) and high (e.g., greater than about 7.5%) ranges of HbA1c. This is likely related to individual variation in red blood cell physiology. These deviations have limited the predictive usefulness of GMI, when compared to measured HbA1c, for making glycemic management plans for individuals with diabetes. For example, measured HbA1c has remained the most useful metric for predicting a patient's likelihood of developing incident DR.

To address these shortcomings, an updated GMI formula has been derived using a kinetic model. A GMI calculation is shown in Equation 2:

$${GMI = ( {PGR}_{ref}^{- 1} * {( AG )}^{- 1} + {K}_{M}^{- 1} ) * 1 {0}^{3} + C ) )}^{- 1} \qquad Equation 2$$

where AG is measured in mg/dL, PGR_{ref} is a reference personal glycation ratio (reference PGR) measured in g/ml (grams per milliliter), K_{M} is the glucose affinity of the glucose transporter type 1 (GLUT1) on red blood cells measured in mg/dl, and C is a constant. In embodiments, PGR_{ref} is in a range from about 50 g/ml to about 80 g/ml, and K_{M} is in a range from about 100 mg/dl to about 1000 mg/dl. In one example, PGR_{ref} is 65.1 g/ml, K_{M} is 473 mg/dl, and C is 0.01. Using these example values (i.e., PGR_{ref}=65.1, K_{M}=473, C=0.01), GMI can be calculated using a simplified formula shown in Equation 3:

$$GMI ( NGSP \% ) = {( \frac{15.36}{AG ( mg / dL )} + 0.0426 )}^{- 1} \qquad Equation 3$$

Using Equations 2 and 3, GMI represents the predicted HbA1c for an individual, given their PGR, with Equation 3 assuming a PGR_{ref} of 65.1 g/ml (likely the average PGR for most individuals). The inventors have discovered that, when calculating GMI using Equation 3, GMI is comparable to HbA1c for making some diagnostic predictions, such as an individual's likelihood of developing incident DR. As a result, inventors have discovered that CGM data alone can be used for such diagnostic predictions, reducing the need for patients to go through the significant inconvenience and cost of obtaining HbA1c tests. This also means that CGM data can be used to gauge an individual's likelihood of developing incident complications, such as DR, on an ongoing basis, rather than based on relatively infrequent HbA1c tests. Further, when GMI calculations are combined with HbA1c measurements, some diagnostic predictions, such as estimating an individual's likelihood of developing incident complications, such as DR, are improved over predictions based on HbA1c measurements alone.

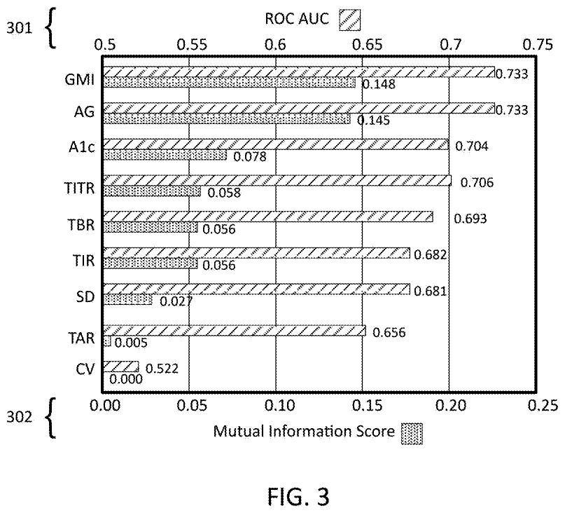

FIG. 3 demonstrates the effectiveness of GMI, HbA1c, and various CGM-derived metrics in making a diagnostic prediction of an individual's likelihood of developing incident DR. The CGM-derived metrics include standard deviation (SD), coefficient of variation (CV), time in range 70-180 mg/dl (TIR), time in tight range 70-140 mg/dl (TITR), time above range >180 mg/dL (TAR), and time below range <70 mg/dl (TBR). In FIG. 3, scale 301 relates to a receiver operating characteristic (ROC) area under the curve (AUC), while scale 302 relates to a mutual information score (MIS). As indicated, ROC AUC values are filled with forward-diagonal shading, while MIS values are filled with solid shading.

Each MIS shown in FIG. 3 indicates the degree of association between a given metric (e.g., HbA1c, GMI, and the CGM-derived metrics) and the occurrence of incident DR. A MIS quantifies how much uncertainty in one variable is reduced by knowing another. As shown in FIG. 3, based on their MISs, GMI demonstrates a stronger association with the occurrence of incident DR (a MIS of 0.148) than HbA1c (a MIS of 0.078). Each ROC AUC shown in FIG. 3 further indicates the discriminative power of a given metric (e.g., HbA1c, GMI, and the CGM-derived metrics) in distinguishing DR from no-DR. A higher ROC AUC value indicates a greater ability of the metric to differentiate between DR and no-DR. As shown in FIG. 3, based on their ROC AUC values, GMI demonstrates a higher discriminative power in distinguishing DR from no-DR (a ROC AUC of 0.733) than HbA1c (a ROC AUC of 0.704).

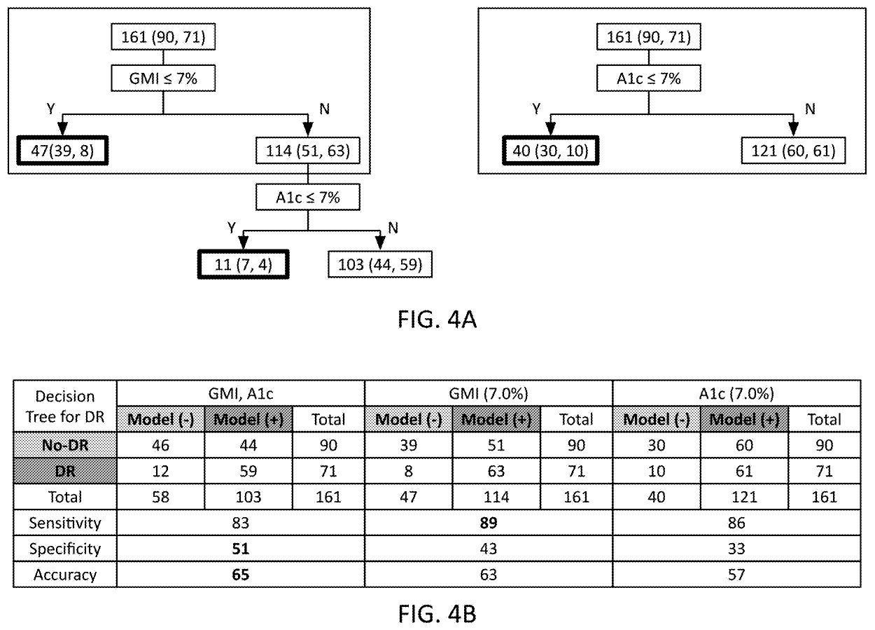

FIGS. 4A and 4B illustrate example decision tree models and performance metrics for predicting DR using GMI and HbA1c. These figures demonstrate the effectiveness of GMI as a predictive tool for incident DR, either as an alternative to HbA1c or in conjunction with it. FIG. 4A presents two decision tree models. The left decision tree utilizes GMI to predict incident DR. Specifically, it shows that for 161 individuals, comprising 90 individuals without DR and 71 with DR, using a GMI≤7% as a prediction model yields 47 individuals being predicted to have no DR (with 39 of those individuals actually having no DR, and 8 of those individuals actually having DR) and using a GMI >7% as a prediction model yields 114 individuals being predicted to have DR (with 51 of those individuals actually having no DR, and 63 of those individuals actually having DR). Adding HbA1c further refines the prediction. For individuals with GMI >7% and HbA1c≤7%, 11 cases were predicted to have no DR (with 7 of those individuals actually having no DR, and 4 of those individuals actually having DR). For individuals with GMI >7% and HbA1c >7%, 103 cases were predicted to have DR (with 44 of those individuals actually having no DR, and 59 of those individuals actually having DR).

The right decision tree uses only HbA1c to predict incident DR. For the same 161 individuals, using an HbA1c threshold of 7% yields the following predictions: for HbA1c≤7%, 40 individuals were predicted to have no DR (with 30 of those individuals actually having no DR, and 10 of those individuals actually having DR), and for HbA1c >7%, 121 individuals were predicted to have DR (with 60 of those individuals actually having no DR, and 61 of those individuals actually having DR).

FIG. 4B compares the performance metrics of three models: GMI+HbA1c, GMI only, and HbA1c only. The combined GMI+HbA1c model achieved a sensitivity of 83%, specificity of 51%, and accuracy of 65%. The GMI only model had a sensitivity of 89%, a specificity of 43%, and an accuracy of 63%. The HbA1c only model showed a sensitivity of 86%, specificity of 33%, and accuracy of 57%. These metrics indicate that GMI alone has a higher sensitivity than HbA1c alone and that combining GMI with HbA1c improves specificity and accuracy, making it a robust tool for predicting incident DR.

### Using GMI to Reduce the Likelihood of Incident Complications

The preceding has shown that CGM-derived GMI values can be utilized to make informed treatment decisions aimed at reducing a diabetic individual's likelihood of incident complications, such as DR, nephropathy, cardiovascular disease, and neuropathy. This means that healthcare providers can use GMI alone or in combination with HbA1c to tailor glycemic treatment plans for individual diabetic patients. If a patient's GMI indicates a high likelihood of developing DR or other complications, the provider may adjust the patient's insulin regimen, recommend dietary changes, recommend fitness changes, and/or prescribe additional medications to better control blood glucose levels. These personalized treatment plans can help maintain glucose levels within a target range, thereby minimizing the likelihood of developing DR and other diabetes-related complications.

In addition to making treatment decisions, GMI can be used to automatically generate local alerts on a user's personal device, such as a smartphone or a dedicated reader device (e.g., reader device 120). For example, if the GMI exceeds a predefined threshold, or exceeds a predefined threshold over a determined amount of time, an alert can be initiated at the user's personal device, prompting them to take action. For example, the user may take immediate action (e.g., administering insulin or consuming glucose), and/or the user may take long-term action (e.g., modifying their dietary and fitness behaviors). In some embodiments, alerts are initiated locally, e.g., by reader device 120. In other embodiments, alerts are initiated remotely, e.g., by trusted computer system 180. In embodiments, alerts are customized based on the patient's specific needs and preferences, ensuring timely interventions that help maintain improved glycemic control. For example, alerts may be customized by a user and/or the user's HCP to adjust the frequency of alerts, to adjust the alerting thresholds (e.g., threshold GMI value, threshold time above a GMI value), or to adjust any recommendations presented in the alerts.

Furthermore, GMI values can trigger alerts to healthcare providers via cloud services, such as trusted computer system 180. When the GMI indicates a likelihood of incident DR or other complications, the system can automatically send notifications to the patient's healthcare provider, allowing them to proactively reach out to the patient and discuss potential adjustments to their treatment plan. This remote monitoring capability ensures that healthcare providers are kept informed of their patients' glycemic status and can intervene promptly to prevent complications.

In various embodiments, GMI values are calculated at a personal device (e.g., reader device 120, a user's smartphone), and/or are calculated by a cloud service (e.g., operating at trusted computer system 180). Similarly, in various embodiments, predictions based on GMI values may be determined at a personal device (e.g., reader device 120, a user's smartphone), and/or by a cloud service (e.g., trusted computer system 180).

In embodiments, analytics are performed at the user's device (e.g., reader device 120) and/or at a cloud service (e.g., trusted computer system 180) to enhance the predictive accuracy and utility of GMI. On the user's device, real-time processing of CGM data can be used to calculate GMI and generate immediate assessments of the likelihood of developing diabetic complications. This local analytics capability allows for quick responses to changes in the patient's glycemic status, providing timely alerts and recommendations. At the cloud service level, more advanced analytics can be conducted using aggregated GMI data from multiple patients. Trusted computer system 180 can leverage machine learning algorithms to continuously update and refine predictive models based on new patient data. These models can improve the accuracy and reliability of the GMI predictions, enabling more precise assessments and better-informed treatment decisions. Additionally, cloud-based analytics can facilitate the generation of comprehensive health reports that summarize the patient's health profile and provide actionable recommendations for both the patient and their healthcare provider.

Some embodiments integrate GMI data into electronic health records (EHR), such as EHRs accessible to the trusted computer system 180. By integrating GMI data into electronic health records, healthcare providers can ensure seamless communication and continuity of care. The GMI data, associated predictions, and alerts can be automatically documented in the patient's EHR, providing a complete and up-to-date record of their glycemic status and health outcome assessments. This integration supports coordinated care efforts, allowing different healthcare providers to access and act upon the same information, ultimately improving patient outcomes.

Moreover, some embodiments integrate GMI data with personal health data platforms such as APPLE HEALTH or GOOGLE FIT, further enhancing the utility GMI. For example, GMI data can be contributed (e.g., by a reader device 120, such as a smartphone) to a health data platform, allowing users to track their GMI alongside other health metrics. This integration enables users to have a comprehensive view of their health data, facilitating better self-management and informed discussions with healthcare providers. Additionally, some embodiments can pull HbA1c data from a health data platform (e.g., which may be contributed to the health data platform by the patient's healthcare organization). This data can be used to refine the on-device or in-cloud analysis, improving the accuracy of GMI-based predictions. By incorporating HbA1c data from these health data platforms, the system can provide more precise health outcome assessments and personalized recommendations, enhancing the overall effectiveness of glycemic management.

Charting overall GMI levels can provide valuable insights into a patient's glycemic trends over time. By visualizing GMI data, patients and healthcare providers can identify patterns and make more informed decisions about glycemic management. For instance, plotting GMI levels over weeks, months, or years can help identify periods of improvement or deterioration, allowing for timely interventions. If a patient's GMI levels are consistently rising, it may indicate the need for adjustments in their treatment plan. In various embodiments, GMI charts, such as GMI trends and historical GMI calculations, are maintained and presented to a user by reader device 120 and/or by a web service (e.g., trusted computer system 180).

GMI charts can also be used to correlate specific behaviors with changes in glycemic control. Patients can track their diet, exercise, and medication adherence alongside their GMI levels, helping them understand how certain behaviors impact their glucose management and motivating them to adopt healthier habits. Healthcare providers can use these charts to set personalized goals for their patients, establishing target GMI ranges and providing regular feedback to help patients stay on track.

Furthermore, GMI charts can serve as an educational tool, helping patients understand the importance of maintaining stable glucose levels. Visualizing the impact of their actions on GMI can reinforce the need for consistent monitoring and adherence to their treatment plan. By leveraging GMI data to guide behavioral interventions, patients can receive personalized recommendations for diet, exercise, and medication, along with real-time feedback on their actions. This immediate feedback can help patients make better choices and stay motivated.

### Example Methods for Using GMI to Reduce the Likelihood of Incident Complications

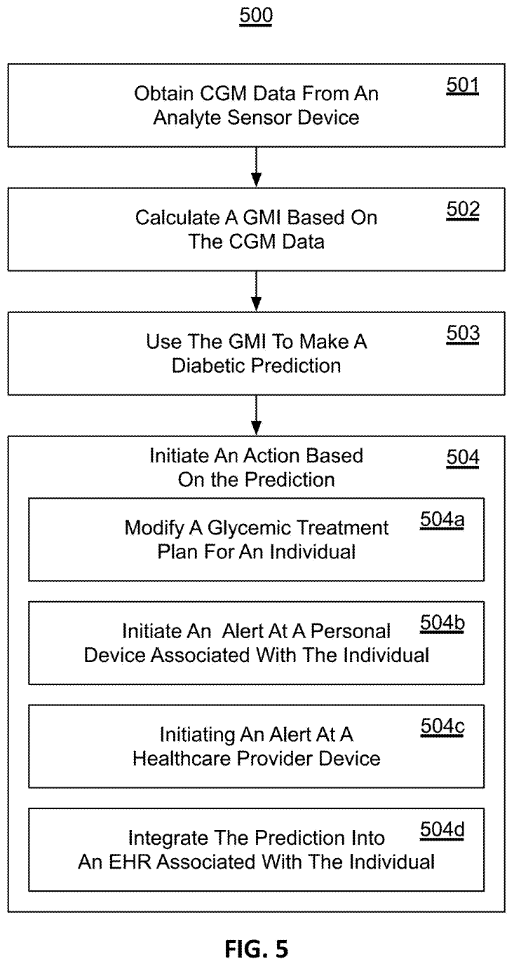

Embodiments are now described in connection with FIG. 5, which illustrates a flow chart of an example method 500 for using GMI to improve glycemic treatment outcomes. In embodiments, instructions for implementing method 500 are encoded as computer-executable instructions stored on a computer-readable storage medium that are executable by a processor to cause a computer system (e.g., reader device 120, local computer system 170 and/or trusted computer system 180) to perform method 500.

The following discussion now refers to a method and method acts. Although the method acts are discussed in specific orders or are illustrated in a flow chart as occurring in a particular order, no order is required unless expressly stated or required because an act is dependent on another act being completed prior to the act being performed.

Referring to FIG. 5, in embodiments, method 500 comprises act 501 of obtaining CGM data from an analyte sensor device. In some embodiments, act 501 comprises obtaining CGM data from an analyte sensor device worn by an individual. For example, reader device 120, local computer system 170, or trusted computer system 180 can obtain CGM data gathered by sensor control device 102 and communicated to reader device 120.

Method 500 also comprises act 502 of calculating a GMI based on the CGM data. In embodiments, the GMI is the GMI described herein. As such, the GMI is derived from AG obtained from the CGM data, PGR_{ref}, and K_{M}. For example, reader device 120, local computer system 170, or trusted computer system 180 can calculate a GMI using Equation 2 or Equation 3, described above, relying on an AG obtained from the CGM data discussed in act 501. In embodiments, PGR_{ref} is in a range from about 50 to about 80, and K_{M} is in a range from about 100 to about 1000.

Method 500 also comprises act 503 of using the GMI to make a diabetic prediction. In some embodiments, act 503 comprises generating a prediction for developing a diabetic complication based on the GMI. For example, reader device 120, local computer system 170, or trusted computer system 180 can make a prediction, such as a prediction of incident DR, based on the calculated GMI. FIGS. 4A and 4B illustrated decision trees for making a prediction of incident DR, based on a GMI that is less than or equal to 7%, though other thresholds may be possible. While DR is provided as an example, the principles described herein may apply to various additional diabetic complications, such as nephropathy, cardiovascular disease, and neuropathy, each with its own GMI threshold for making predictions.

As illustrated in FIG. 4A, such a prediction may be based on GMI alone, or based on a combination of GMI and HbA1c. Thus, in some embodiments, the method can include obtaining an HbA1c measurement for the individual (e.g., from an EHR, from a health data platform, from a manual user entry) and refining the prediction by combining the GMI with the HbA1c measurement. In embodiments, when combining a GMI with an HbA1c measurement, the HbA1c measurement is obtained within approximately thirty days of a glucose collection period used to calculate the GMI. Preferably, the HbA1c measurement is obtained at the end of this glucose collection period. Additionally, some embodiments utilize a weighting factor based on the age of an HbA1c measurement when creating a combined GMI/HbA1c prediction. For example, a more recent HbA1c measurement may have a higher weighting than a less recent HbA1c measurement, meaning that a more recent HbA1c measurement may contribute to a combined GMI/HbA1c prediction more than a less recent HbA1c measurement.

Method 500 also comprises act 504 of initiating an action based on the prediction. As shown, in embodiments, the action is selected from a group consisting of modifying a glycemic treatment plan for the individual (act 504a), initiating an alert at a personal device associated with the individual (act 504b), initiating an alert at a healthcare provider device (act 504c), and integrating the prediction into an EHR associated with the individual (act 504d). For example, reader device 120, local computer system 170, or trusted computer system 180 can initiate an action when there is a prediction of an incident diabetic condition based on the GMI value.

In some embodiments, when the action is initiating an alert at the personal device associated with the individual, or when the action is initiating an alert at the healthcare provider device, the alert is based on the GMI exceeding a predefined threshold, or exceeding the predefined threshold over a determined amount of time.

In some embodiments, when the action is integrating the prediction into an EHR associated with the individual, the method also comprises integrating the GMI into the EHR.

Alternatively or in addition to the other examples described herein, examples include any combination of the following:

Clause 1. A method, implemented in a computer system, comprising: obtaining continuous glucose monitoring (CGM) data from an analyte sensor device worn by an individual; calculating a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M}); generating a prediction for developing a diabetic complication based on the GMI; and initiating an action based on the prediction, wherein the action is selected from a group consisting of: modifying a glycemic treatment plan for the individual, initiating a first alert at a personal device associated with the individual, initiating a second alert at a healthcare provider device, and integrating the prediction into an electronic health record (EHR) associated with the individual.

Clause 2. The method of clause 1, wherein the action is modifying the glycemic treatment plan for the individual.

Clause 3. The method of clause 1, wherein the action is initiating the first alert at the personal device associated with the individual.

Clause 4. The method of clause 3, wherein the first alert is based on the GMI exceeding a predefined threshold, or exceeding the predefined threshold over a determined amount of time.

Clause 5. The method of clause 1, wherein the action is initiating the second alert at the healthcare provider device.

Clause 6. The method of clause 3, wherein the second alert is based on the GMI exceeding a predefined threshold, or exceeding the predefined threshold over a determined amount of time.

Clause 7. The method of clause 1, wherein the action is integrating the prediction into the EHR associated with the individual.

Clause 8. The method of any of clauses 1 to 7, wherein the diabetic complication is selected from a group consisting of diabetic retinopathy, nephropathy, cardiovascular disease, and neuropathy.

Clause 9. The method of any of clauses 1 to 8, wherein the method further comprises integrating the GMI into the EHR.

Clause 10. The method of any of clauses 1 to 9, wherein the method further comprises: obtaining an HbA1c measurement for the individual; and refining the prediction by combining the GMI with the HbA1c measurement.

Clause 11. The method of clause 10, wherein obtaining the HbA1c measurement for the individual comprises pulling the HbA1c measurement from a health data platform.

Clause 12. The method of any of clauses 1 to 11, wherein the method further comprises contributing the GMI to a health data platform.

Clause 13. The method of any of clauses 1 to 12, wherein the method further comprises contributing the prediction to a health data platform.

Clause 14. The method of any of clauses 11 to 13, wherein the health data platform is APPLE HEALTH or GOOGLE FIT.

Clause 15. The method of any of clauses 1 to 14, wherein modifying the glycemic treatment plan comprises at least one of: adjusting an insulin regimen; recommending a dietary change; prescribing an additional medication; setting a personalized glucose target; or setting a monitoring schedule.

Clause 16. The method of any of clauses 1 to 15, wherein calculating the GMI comprises using a formula,

$${GMI = ( {PGR}_{ref}^{- 1} * {( AG )}^{- 1} + {K}_{M}^{- 1} ) * 1 {0}^{3} + C ) )}^{- 1} ,$$

where AG is measured in mg/dl, PGR_{ref} is measured in g/ml, and K_{M} is measured in mg/dL.

Clause 17. The method of any of clauses 1 to 16, wherein PGR_{ref} is in a range from about 50 to about 80, and K_{M} is in a range from about 100 to about 1000.

Clause 18. The method of any of clauses 1 to 17, wherein calculating the GMI comprises using a formula,

$$GMI = {( \frac{15.36}{AG} + 0.0426 )}^{- 1} ,$$

where AG is measured in mg/dL.

Clause 19. The method of any of clauses 1 to 18, wherein the computer system is a reader device.

Clause 20. The method of any of clauses 1 to 18, wherein the computer system operates a cloud service.

Clause 21. A computer system, comprising: a processor; and a computer-readable storage medium that stores computer-executable instructions that are executable by the processor to at least: obtain continuous glucose monitoring (CGM) data from an analyte sensor device worn by an individual; calculate a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M}); generate a prediction for developing a diabetic complication based on the GMI; and initiate an action based on the prediction, wherein the action is selected from a group consisting of: modifying a glycemic treatment plan for the individual, initiating a first alert at a personal device associated with the individual, initiating a second alert at a healthcare provider device, and integrating the prediction into an electronic health record (EHR) associated with the individual.

Clause 22. The computer system of clause 21, wherein the action is modifying the glycemic treatment plan for the individual.

Clause 23. The computer system of clause 21, wherein the action is initiating the first alert at the personal device associated with the individual.

Clause 24. The computer system of clause 23, wherein the first alert is based on the GMI exceeding a predefined threshold, or exceeding the predefined threshold over a determined amount of time.

Clause 25. The computer system of clause 21, wherein the action is initiating the second alert at the healthcare provider device.

Clause 26. The computer system of clause 25, wherein the second alert is based on the GMI exceeding a predefined threshold, or exceeding the predefined threshold over a determined amount of time.

Clause 27. The computer system of clause 21, wherein the action is integrating the prediction into the EHR associated with the individual.

Clause 28. The computer system of any of clauses 21 to 27, wherein the diabetic complication is selected from a group consisting of diabetic retinopathy, nephropathy, cardiovascular disease, and neuropathy.

Clause 29. The computer system of any of clauses 21 to 28, wherein the computer-executable instructions are executable by the processor to integrate the GMI into the EHR.

Clause 30. The computer system of any of clauses 21 to 29, wherein the computer-executable instructions are executable by the processor to: obtain an HbA1c measurement for the individual; and refine the prediction by combining the GMI with the HbA1c measurement.

Clause 31. The computer system of clause 30, wherein obtaining the HbA1c measurement for the individual comprises pulling the HbA1c measurement from a health data platform.

Clause 32. The computer system of any of clauses 21 to 31, the computer-executable instructions are executable by the processor to contribute the GMI to a health data platform.

Clause 33. The computer system of any of clauses 21 to 32, the computer-executable instructions are executable by the processor to contribute the prediction to a health data platform.

Clause 34. The computer system of any of clauses 31 to 33, wherein the health data platform is APPLE HEALTH or GOOGLE FIT.

Clause 35. The computer system of any of clauses 21 to 34, wherein modifying the glycemic treatment plan comprises at least one of: adjusting an insulin regimen; recommending a dietary change; prescribing an additional medication; setting a personalized glucose target; or setting a monitoring schedule.

Clause 36. The computer system of any of clauses 21 to 35, wherein calculating the GMI comprises using a formula,

$${GMI = ( {PGR}_{ref}^{- 1} * {( AG )}^{- 1} + {K}_{M}^{- 1} ) * 1 {0}^{3} + C ) )}^{- 1} ,$$

where AG is measured in mg/dL, PGR_{ref} is measured in g/ml, and K_{M} is measured in mg/dL.

Clause 37. The computer system of any of clauses 21 to 36, wherein PGR_{ref} is in a range from about 50 to about 80, and K_{M} is in a range from about 100 to about 1000.

Clause 38. The computer system of any of clauses 21 to 37, wherein calculating the GMI comprises using a formula,

$$GMI = {( \frac{15.36}{AG} + 0.0426 )}^{- 1} ,$$

where AG is measured in mg/dL.

Clause 39. The computer system of any of clauses 20 to 36, wherein the computer system is a reader device.

Clause 40. The computer system of any of clauses 20 to 36, wherein the computer system operates a cloud service.

Clause 41. A computer-readable storage medium that stores computer-executable instructions that are executable by a processor to at least: obtain continuous glucose monitoring (CGM) data from an analyte sensor device worn by an individual; calculate a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M}); generate a prediction for developing a diabetic complication based on the GMI; and initiate an action based on the prediction, wherein the action is selected from a group consisting of: modifying a glycemic treatment plan for the individual, initiating a first alert at a personal device associated with the individual, initiating a second alert at a healthcare provider device, and integrating the prediction into an electronic health record (EHR) associated with the individual.

Clause 42. The computer-readable storage medium of clause 41, wherein the action is modifying the glycemic treatment plan for the individual.

Clause 43. The computer-readable storage medium of clause 41, wherein the action is initiating the first alert at the personal device associated with the individual.

Clause 44. The computer-readable storage medium of clause 41, wherein the first alert is based on the GMI exceeding a predefined threshold, or exceeding the predefined threshold over a determined amount of time.

Clause 45. The computer-readable storage medium of clause 41, wherein the action is initiating the second alert at the healthcare provider device.

Clause 46. The computer-readable storage medium of clause 45, wherein the second alert is based on the GMI exceeding a predefined threshold, or exceeding the predefined threshold over a determined amount of time.

Clause 47. The computer-readable storage medium of clause 41, wherein the action is integrating the prediction into the EHR associated with the individual.

Clause 48. The computer-readable storage medium of any of clauses 41 to 47, wherein the diabetic complication is selected from a group consisting of diabetic retinopathy, nephropathy, cardiovascular disease, and neuropathy.

Clause 49. The computer-readable storage medium of any of clauses 41 to 48, wherein the computer-executable instructions are executable by the processor to integrate the GMI into the EHR.

Clause 50. The computer-readable storage medium of any of clauses 41 to 49, wherein the computer-executable instructions are executable by the processor to: obtain an HbA1c measurement for the individual; and refine the prediction by combining the GMI with the HbA1c measurement.

Clause 51. The computer-readable storage medium of clause 50, wherein obtaining the HbA1c measurement for the individual comprises pulling the HbA1c measurement from a health data platform.

Clause 52. The computer-readable storage medium of any of clauses 41 to 51, wherein the computer-executable instructions are executable by the processor to contribute the GMI to a health data platform.

Clause 53. The computer-readable storage medium of any of clauses 41 to 52, wherein the computer-executable instructions are executable by the processor to contribute the prediction to a health data platform.

Clause 54. The computer-readable storage medium of any of clauses 51 to 53, wherein the health data platform is APPLE HEALTH or GOOGLE FIT.

Clause 55. The computer-readable storage medium of any of clauses 41 to 54, wherein modifying the glycemic treatment plan comprises at least one of: adjusting an insulin regimen; recommending a dietary change; prescribing an additional medication; setting a personalized glucose target; or setting a monitoring schedule.

Clause 56. The computer-readable storage medium of any of clauses 41 to 55, wherein calculating the GMI comprises using a formula,

$${GMI = ( {PGR}_{ref}^{- 1} * {( AG )}^{- 1} + {K}_{M}^{- 1} ) * 1 {0}^{3} + C ) )}^{- 1} ,$$

where AG is measured in mg/dL, PGR_{ref} is measured in g/ml, and K_{M} is measured in mg/dL.

Clause 57. The computer-readable storage medium of any of clauses 41 to 56, wherein PGR_{ref} is in a range from about 50 to about 80, and K_{M} is in a range from about 100 to about 1000.

Clause 58. The computer-readable storage medium of any of clauses 41 to 57, wherein calculating the GMI comprises using a formula,

$$GMI = {( \frac{15.36}{AG} + 0.0426 )}^{- 1} ,$$

where AG is measured in mg/dL.

Embodiments of the disclosure comprise or utilize a special-purpose or general-purpose computer system that includes computer hardware, such as, for example, a processor system and system memory, as discussed in greater detail below. Embodiments within the scope of the present disclosure also include physical and other computer-readable media for carrying or storing computer-executable instructions and/or data structures. Such computer-readable media can be any available media accessible by a general-purpose or special-purpose computer system. Computer-readable media that store computer-executable instructions and/or data structures are computer storage media. Computer-readable media that carry computer-executable instructions and/or data structures are transmission media. Thus, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as random access memory (RAM), read-only memory (ROM), electrically erasable programmable ROM (EEPROM), solid state drives (SSDs), flash memory, phase-change memory (PCM), optical disk storage, magnetic disk storage or other magnetic storage devices, or any other hardware storage device(s) which store program code in the form of computer-executable instructions or data structures, which can be accessed and executed by a general-purpose or special-purpose computer system to implement the disclosed functionality.

Transmission media include a network and/or data links that carry program code in the form of computer-executable instructions or data structures that are accessible by a general-purpose or special-purpose computer system. A “network” is defined as a data link that enables the transport of electronic data between computer systems and other electronic devices. When information is transferred or provided over a network or another communications connection (either hardwired, wireless, or a combination thereof) to a computer system, the computer system may view the connection as transmission media. The scope of computer-readable media includes combinations thereof.

Upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module and eventually transferred to computer system RAM and/or less volatile computer storage media at a computer system. Thus, computer storage media can be included in computer system components that also utilize transmission media.

Computer-executable instructions comprise, for example, instructions and data which when executed at a processor system, cause a general-purpose computer system, a special-purpose computer system, or a special-purpose processing device to perform a function or group of functions. In embodiments, computer-executable instructions comprise binaries, intermediate format instructions (e.g., assembly language), or source code. In embodiments, a processor system comprises one or more central processing units (CPUs), one or more graphics processing units (GPUs), one or more neural processing units (NPUs), and the like.

In some embodiments, the disclosed systems and methods are practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. In some embodiments, the disclosed systems and methods are practiced in distributed system environments where different computer systems, which are linked through a network (e.g., by hardwired data links, wireless data links, or by a combination of hardwired and wireless data links), both perform tasks. As such, in a distributed system environment, a computer system may include a plurality of constituent computer systems. Program modules may be located in local and remote memory storage devices in a distributed system environment.

In some embodiments, the disclosed systems and methods are practiced in a cloud computing environment. In some embodiments, cloud computing environments are distributed, although this is not required. When distributed, cloud computing environments may be distributed internally within an organization and/or have components possessed across multiple organizations. In this description and the following claims, “cloud computing” is a model for enabling on-demand network access to a shared pool of configurable computing resources (e.g., networks, servers, storage, applications, and services). A cloud computing model can be composed of various characteristics, such as on-demand self-service, broad network access, resource pooling, rapid elasticity, measured service, and so forth. A cloud computing model may also come in the form of various service models such as Software as a Service (Saas), Platform as a Service (PaaS), Infrastructure as a Service (IaaS), etc. The cloud computing model may also be deployed using different deployment models such as private cloud, community cloud, public cloud, hybrid cloud, etc.

Some embodiments, such as a cloud computing environment, comprise a system with one or more hosts capable of running one or more virtual machines (VMs). During operation, VMs emulate an operational computing system, supporting an operating system (OS) and perhaps one or more other applications. In some embodiments, each host includes a hypervisor that emulates virtual resources for the VMs using physical resources that are abstracted from the view of the VMs. The hypervisor also provides proper isolation between the VMs. Thus, from the perspective of any given VM, the hypervisor provides the illusion that the VM is interfacing with a physical resource, even though the VM only interfaces with the appearance (e.g., a virtual resource) of a physical resource. Examples of physical resources include processing capacity, memory, disk space, network bandwidth, media drives, and so forth.

Although the subject matter has been described in language specific to structural features and/or methodological acts, it is to be understood that the subject matter defined in the appended claims is not necessarily limited to the described features or acts described supra or the order of the acts described supra. Rather, the described features and acts are disclosed as example forms of implementing the claims.

The present disclosure may be embodied in other specific forms without departing from its essential characteristics. The described embodiments are only illustrative and not restrictive. All changes that come within the meaning and range of equivalency of the claims are to be embraced within their scope.

When introducing elements in the appended claims, the articles “a,” “an,” “the,” and “said” are intended to mean there are one or more of the elements. The terms “comprising,” “including,” and “having” are intended to be inclusive and mean that there may be additional elements other than the listed elements. Unless otherwise specified, the terms “set,” “superset,” and “subset” are intended to exclude an empty set, and thus “set” is defined as a non-empty set, “superset” is defined as a non-empty superset, and “subset” is defined as a non-empty subset. Unless otherwise specified, the term “subset” excludes the entirety of its superset (i.e., the superset contains at least one item not included in the subset). Unless otherwise specified, a “superset” can include at least one additional element, and a “subset” can exclude at least one element.

What is claimed:

1. A method, implemented in a computer system, comprising:
obtaining continuous glucose monitoring (CGM) data from an analyte sensor device worn by an individual;
calculating a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M});
generating a prediction for developing a diabetic complication based on the GMI; and
initiating an action based on the prediction, wherein the action is selected from a group consisting of:
modifying a glycemic treatment plan for the individual,
initiating a first alert at a personal device associated with the individual,
initiating a second alert at a healthcare provider device, and
integrating the prediction into an electronic health record (EHR) associated with the individual.

2. The method of claim 1, wherein the action is modifying the glycemic treatment plan for the individual.

3. The method of claim 1, wherein the action is initiating the first alert at the personal device associated with the individual.

4. The method of claim 3, wherein the first alert is based on the GMI exceeding a predefined threshold, or exceeding the predefined threshold over a determined amount of time.

5. The method of claim 1, wherein the action is initiating the second alert at the healthcare provider device.

6. The method of claim 3, wherein the second alert is based on the GMI exceeding a predefined threshold, or exceeding the predefined threshold over a determined amount of time.

7. The method of claim 1, wherein the action is integrating the prediction into the EHR associated with the individual.

8. The method of claim 1, wherein the diabetic complication is selected from a group consisting of diabetic retinopathy, nephropathy, cardiovascular disease, and neuropathy.

9. The method of claim 1, wherein the method further comprises integrating the GMI into the EHR.

10. The method of claim 1, wherein the method further comprises:
obtaining an HbA1c measurement for the individual; and
refining the prediction by combining the GMI with the HbA1c measurement.

11. The method of claim 10, wherein obtaining the HbA1c measurement for the individual comprises pulling the HbA1c measurement from a health data platform.

12. The method of claim 1, wherein the method further comprises contributing at least one of the GMI or the prediction to a health data platform.

13. The method claim 1, wherein modifying the glycemic treatment plan comprises at least one of:
adjusting an insulin regimen;
recommending a dietary change;
prescribing an additional medication;
setting a personalized glucose target; or
setting a monitoring schedule.

14. The method of claim 1, wherein calculating the GMI comprises using a formula,
$${GMI = ( {PGR}_{ref}^{- 1} * {( AG )}^{- 1} + {K}_{M}^{- 1} ) * 1 {0}^{3} + C ) )}^{- 1} ,$$
where AG is measured in mg/dl, PGR_{ref} is measured in g/ml, and K_{M} is measured in mg/dL.

15. The method of claim 1, wherein PGR_{ref} is in a range from about 50 to about 80, and K_{M} is in a range from about 100 to about 1000.

16. The method of claim 1, wherein calculating the GMI comprises using a formula,
$$GMI = {( \frac{15.36}{AG} + 0.0426 )}^{- 1} ,$$
where AG is measured in mg/dL.

17. The method of claim 1, wherein the computer system is a reader device.

18. The method of claim 1, wherein the computer system operates a cloud service.

19. A computer system, comprising:
a processor; and
a computer-readable storage medium that stores computer-executable instructions that are executable by the processor to at least:
obtain continuous glucose monitoring (CGM) data from an analyte sensor device worn by an individual;
calculate a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M});
generate a prediction for developing a diabetic complication based on the GMI; and
initiate an action based on the prediction, wherein the action is selected from a group consisting of:
modifying a glycemic treatment plan for the individual,
initiating a first alert at a personal device associated with the individual,
initiating a second alert at a healthcare provider device, and
integrating the prediction into an electronic health record (EHR) associated with the individual.

20. A non-transitory computer-readable storage medium that stores computer-executable instructions that are executable by a processor to at least:
obtain continuous glucose monitoring (CGM) data from an analyte sensor device worn by an individual;
calculate a glucose management indicator (GMI) based on the CGM data, wherein the GMI is derived from an average sensor glucose (AG) obtained from the CGM data, a reference personal glycation ratio (PGR_{ref}), and a glucose affinity of Glucose Transporter Type-1 on red blood cells (K_{M});
generate a prediction for developing a diabetic complication based on the GMI; and
initiate an action based on the prediction, wherein the action is selected from a group consisting of:
modifying a glycemic treatment plan for the individual,
initiating a first alert at a personal device associated with the individual,
initiating a second alert at a healthcare provider device, and
integrating the prediction into an electronic health record (EHR) associated with the individual.
